(19) United States
(12) Patent Application Publication
(10) Pub. No.: US 2024/0156602 A1
(43) Pub. Date: May 16, 2024

(54) OSTEOTOMY GAP FILLING IMPLANT AND METHODS

(71) Applicant: PORIFEROUS, LLC, Newnan, GA (US)

(72) Inventors: Aaron Noble, Newnan, GA (US); Spencer Jones, Newnan, GA (US)

(73) Assignee: PORIFEROUS, LLC, Newnan, GA (US)

(21) Appl. No.: 18/508,059

(22) Filed: Nov. 13, 2023

Related U.S. Application Data
(60) Provisional application No. 63/383,564, filed on Nov. 14, 2022.

(51) Int. Cl. A61F 2/28

(52) U.S. Cl. CPC A61F 2/2875; A61F 2002/30736

(57) ABSTRACT

An osteotomy gap filling implants and related methods. In one example, the implant may include several implant segments, at least some of which include a porous body, in combination with a non-porous flexible spine extending through the implant segments.

### CROSS-REFERENCE TO RELATED APPLICATIONS

This patent application claims priority to and the benefit of the filing date of U.S. provisional patent application Ser. No. 63/383,564 filed Nov. 14, 2022 for “Osteotomy Gap Filling Implant And Methods,” the entire contents of which are hereby incorporated by this reference.

### RELATED FIELDS

Osteotomy gap filling implants such as implants for filling bone gaps resulting from craniotomies, cranial fixation systems of bone flaps and fractures, and related methods.

### BACKGROUND

Osteotomy procedures can leave bone gaps. In many instances it is desirable to fill those bone gaps. For example, cranial osteotomy procedures often involve cutting a cranial bone flap that leaves a kerf that needs to be filled in order to facilitate reconnecting the blood supply and osteointegration to the cranial flap as well as to improve patient post-procedure cosmesis. Gap with fixation plating provides the surgeon with a novel system that address both the kerf width created when the bone is cut and fixation. See the abandoned patent application US 2010/0036413 A1 as an example of a prior art gap filling device.

The present invention solves the problems associated with the gap filling device freely articulating in segments that allow for smooth transition.

FIG. 1 schematically shows an example of a cranial osteotomy in which a bone flap 102 has been cut from the surrounding cranial bone 104. As shown in FIG. 1, the cranial osteotomy leaves a burr hole 106 (where a hole was drilled to allow access by the osteotome) and a kerf 108 (from the osteotome cutting the bone flap 102). The resulting kerf 108 is semicircular and typically approximately 1-5 mm in width. Depending on whether and how the bone flap 102 is repositioned relative to the surrounding cranial bone 104, the kerf 108 may taper from a wider end 110 of the kerf 108 to a narrower end 112.

The implants that have been used in the past for cranial osteotomy kerfs leave room for improvement. For instance, Medpor porous polyethylene implants have been considered previously for filling cranial osteotomy kerfs. See Duck-Ho Goh, M. D. et al., “Medpor Craniotomy Gap Wedge Designed to Fill Small Bone Defects along Cranial Bone Flap” J Korean Neurosug Soc 46: 195-198, 2009. Problematically, when these previous implants are bent to fit the kerf's curvature, the implants buckle, reducing aesthetic results and otherwise negatively impacting on post-operative results. These and other previous implants may also be prone to breaking in the operating room when bent. The current state of the art leaves significant room for improvement.

### SUMMARY

Described herein are examples of osteotomy gap filling implants and methods. The osteotomy gap filling implant may include several implant segments, at least some of which are formed of a porous body, with a non-porous flexible spine extending through the plurality of implant segments.

In at least some implementations, the implant may be freely bent in several directions without the undesirable buckling that occurs in earlier implant designs. In some implementations, segments of the implants described herein bend about weakened joints that align with the center of nested radial plates.

In at least some implementations, the non-porous flexible spine strengthens the implant while remaining small enough not to prevent tissue and vascular structures from integrating through the implant to facilitate reconnecting the blood supply and osteointegration.

In one example an osteotomy gap filling implant includes implant segments, with at least some of the implant segments including a porous body. The implant also includes a non-porous flexible spine extending through the implant segments.

In this example the adjacent implant segments may be configured to flex relative to one another about at least one axis.

In this example the adjacent implant segments may be configured to flex relative to one another about multiple axes.

In this example the porous bodies of at least some of the implant segments may each include a top plate and a lower body descending from the top plate.

In this example the top plates of the porous bodies may be a series of radially nested top plates.

In this example the non-porous flexible spine may extend through the lower bodies.

In this example a first top plate of one of the implant segments and a second top plate of an adjacent implant segment may be at least partially separated by a curved gap defined by a first curved edge of the first top plate and a second curved edge of the second top plate.

In this example the first curved edge may be a convex curved edge and the second curved edge may be a concave curved edge corresponding in shape to the convex curved edge.

In this example a first lower body of one of the implant segments and a second lower body of an adjacent implant segment may be at least partially separated by a vertical break.

In this example the vertical break between the first and second lower bodies may define a flexural gap between the first and second lower bodies.

In this example at least one of the lower bodies at a first end of the implant may have a narrower width relative to one of the lower bodies at a second end of the implant.

In this example the lower bodies may taper in width from a first end of the implant to a second end of the implant.

In this example the flexible spine may be a single piece spine.

In this example the flexible spine may be a multi-piece spine.

In this example the multi-piece spine may include several rigid spine segments joined at articular joints.

In this example the articular joints may include ball and socket joints.

In this example at least one of the implant segments may also include an insert.

In this example the segment with the insert may have either a porous body or a non-porous body.

In this example the insert may be either entirely embedded in the segment or partially embedded in the segment.

In this example the insert may have holes or not have holes.

In this example the insert may be metal and the implant segment may be plastic.

In this example at least one of the implant segments may include a burr hole cover with a top plate that is larger than top plates of adjacent implant segments.

In this example at least some of the implant segments may be a sintered material molded to the non-porous flexible spine.

In this example the implant may be a cranial osteotomy gap filling implant.

In this example the spine may be configured to be elongated in length by at least 100% without breaking.

In this example the spine may be configured to be elongated in length by at least 300% without breaking.

In another example an osteotomy gap filling method may include steps of: (a) performing a cranial osteotomy in a cranial bone to form a kerf in the cranial bone; and (b) implanting an osteotomy gap filling implant into the kerf, the implant including: (i) several implant segments, at least some of the segments including a porous body; and (ii) a non-porous flexible spine extending through the implant segments.

In this example forming the kerf may constitute forming a curved kerf in the cranial bone.

In this example adjacent implant segments may be configured to flex relative to one another about multiple axes such that the implant can be curved to match the curved kerf in the cranial bone.

In this example the porous bodies of at least some of the implant segments each may include a top plate and a lower body descending from the top plate, the top plates of the porous bodies constituting a series of radially nested top plates.

In this example, during curving the implant to match the curved kerf, the spine may be configured to be elongated in length by at least 100% without breaking.

In this example, during curving the implant to match the curved kerf, the spine may be configured to be elongated in length by at least 300% without breaking.

In this example, prior to implanting the osteotomy gap filling implant into the kerf, the method may include repositioning a portion of the cranial bone such that the kerf tapers from a wider end to a narrower end.

In this example the porous bodies of at least some of the implant segments each may include a top plate and a lower body descending from the top plate, at least one of the lower bodies at a first end of the implant having a narrower width relative to one of the lower bodies at a second end of the implant. The method may be performed such that the implant is implanted into the kerf such that the first end of the implant is proximate the narrower end of the kerf and the second end of the implant is proximate the wider end of the kerf.

### BRIEF DESCRIPTION OF FIGURES

FIG. 1 schematically illustrates a cranial osteotomy.

FIGS. 2a-e illustrate an example of an osteotomy gap filling implant.

FIGS. 3a-e illustrate another example of an osteotomy gap filling implant.

FIGS. 4-12 illustrate additional examples of osteotomy gap filling implants.

FIG. 13 illustrates an example of osteotomy gap filling implants implanted into a cranial bone.

FIG. 14 is a cross section illustrating how lower bodies of an osteotomy gap filling implant may taper in width from one end of the implant to the other end of the implant to match a tapered kerf in a cranial bone.

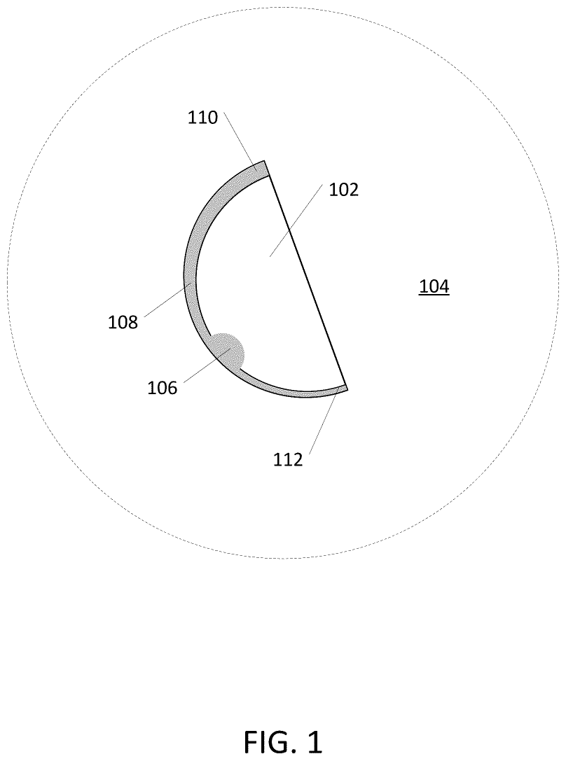

### DETAILED DESCRIPTION

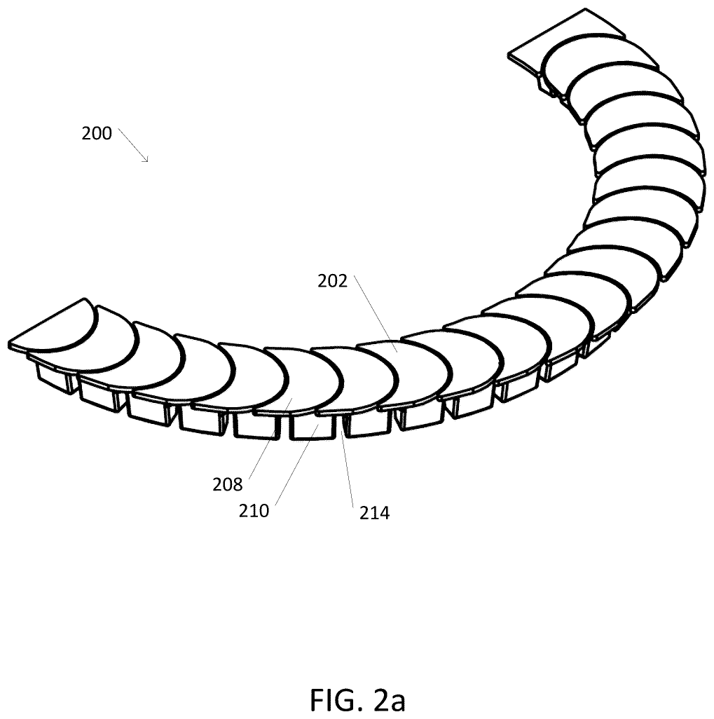

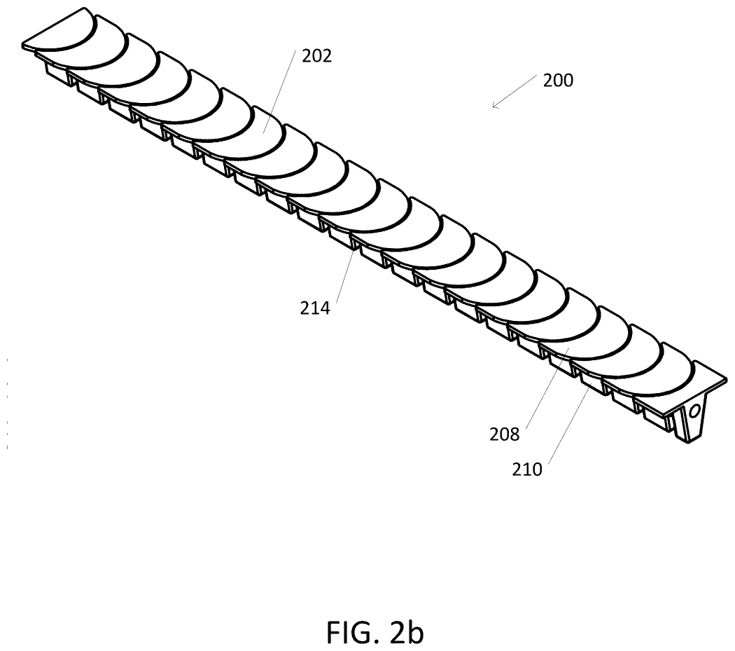

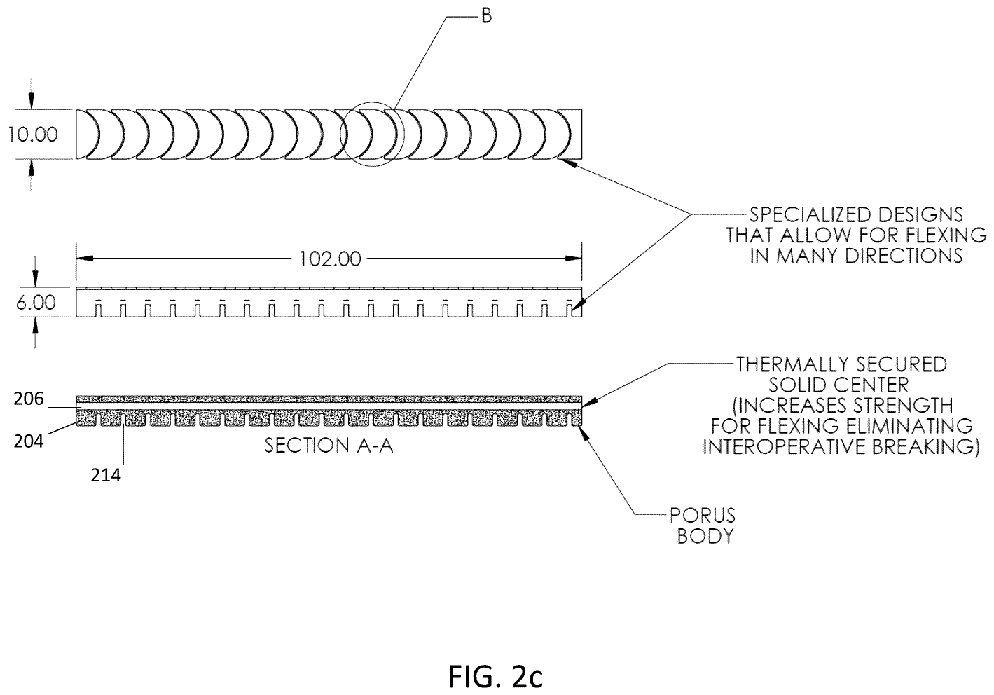

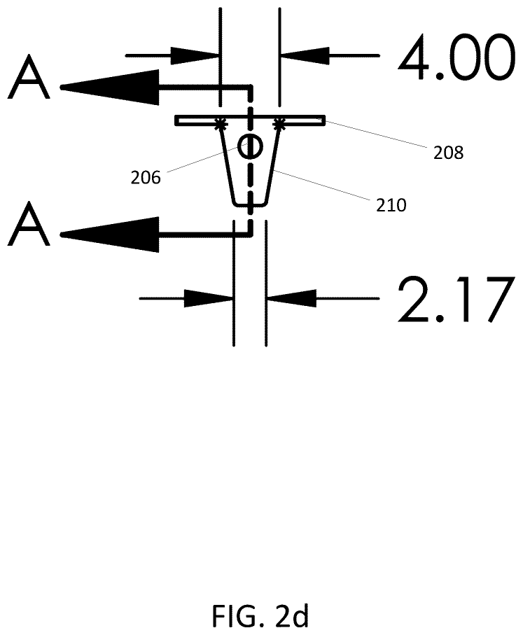

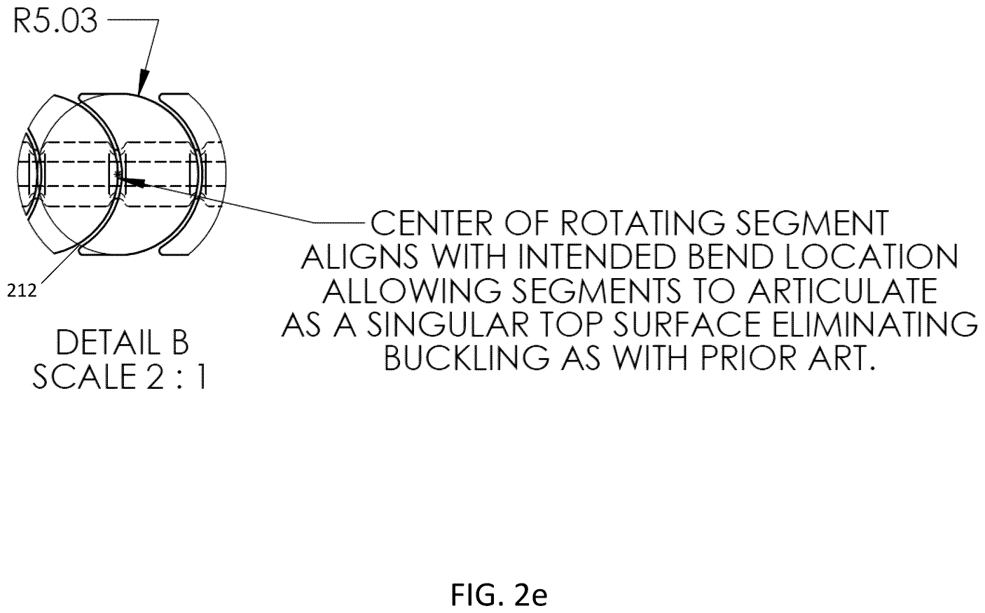

FIGS. 2a-e illustrate one example of an osteotomy gap filling implant 200. In this example, the implant 200 includes several implant segments 202 formed by porous bodies 204 and a non-porous flexible spine 206 extending through the implant segments 202 (see FIG. 2c). The non-porous flexible spine 206 in this example may be a solid material (e.g. a strip or rod of solid polyethylene or other flexible plastic or other material that is formed by compression or injection molding) with the bodies 204 formed by a sintered material (e.g. polyethylene particles) that have been sintered and over-molded onto the spine 206. As shown and discussed in context of other examples presented below, the porous bodies 204 and/or spine 206 may be made of other materials and formed in other manners.

In the examples presented herein, the implants are cranial osteotomy gap filling implants. In other implementations, the implants may be configured for use in other craniotomy procedures, surgery of the orbit, surgery of the craniomaxillofacial areas, and other areas of the body where bone is cut and reconnection is needed.

In the examples presented herein, the adjacent implant segments are configured to flex relative to one another about at least one axis, and in these particular examples, they are configured to flex relative to one another about multiple axes.

In FIGS. 2a-e, the porous bodies 204 of each segment 202 each include a top plate and a lower body descending from the top plate. In this example, the non-porous flexible spine 206 extends through the lower bodies 210.

As can be seen in FIGS. 2a-c and 2e, the top plates 208 form a series of radially nested top plates. More particularly, as can be seen best in FIG. 2e, adjacent top plates 208 are separated by a curved gap 212 defined by the curved edges of adjacent plates 208, with adjacent curved edges being convex and concave in corresponding shapes to one another.

As can be seen in FIG. 2c, the lower bodies 210 are partially separated from one another by vertical breaks 214 that define flexural gaps between adjacent bodies 204.

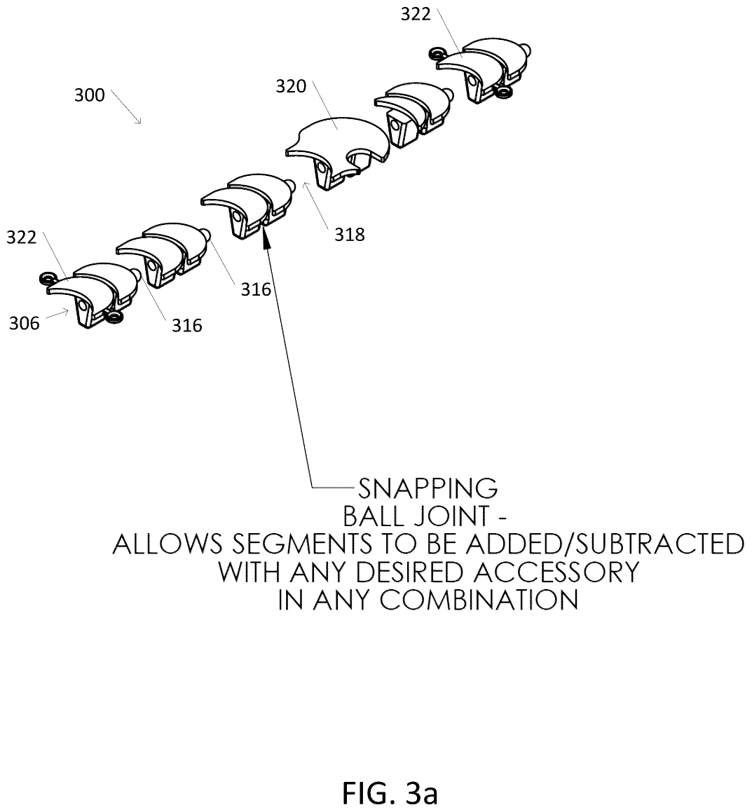

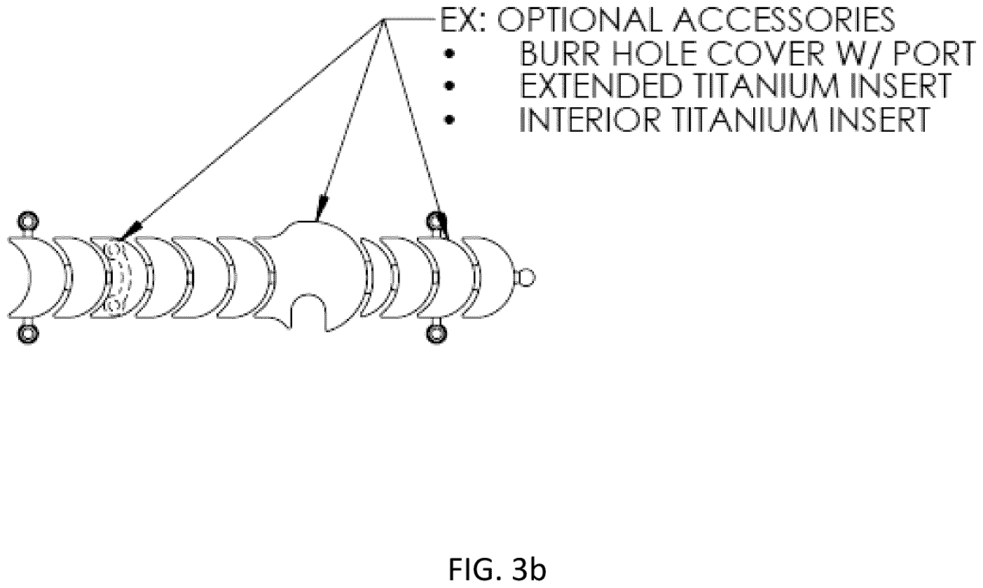

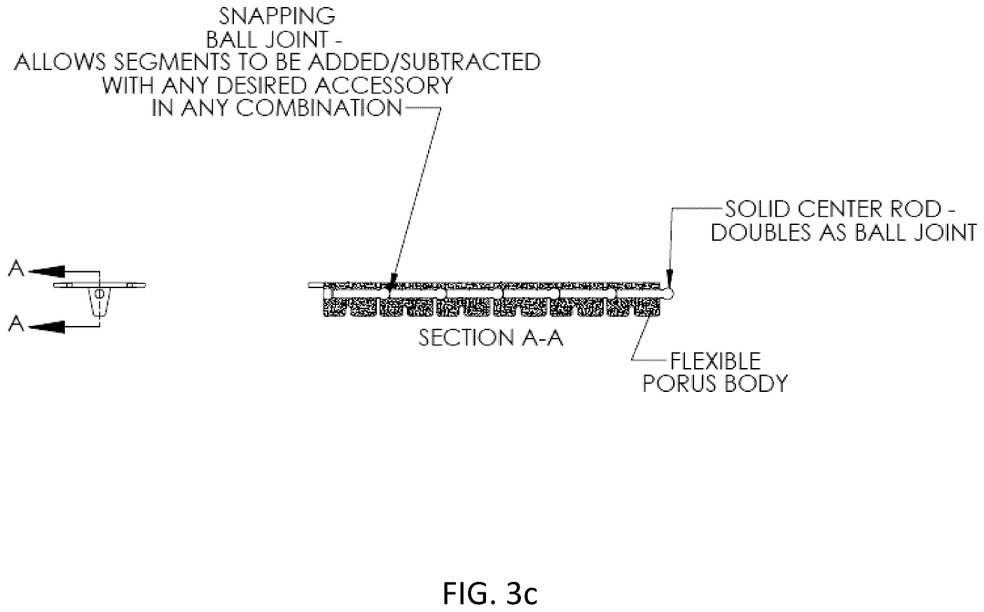

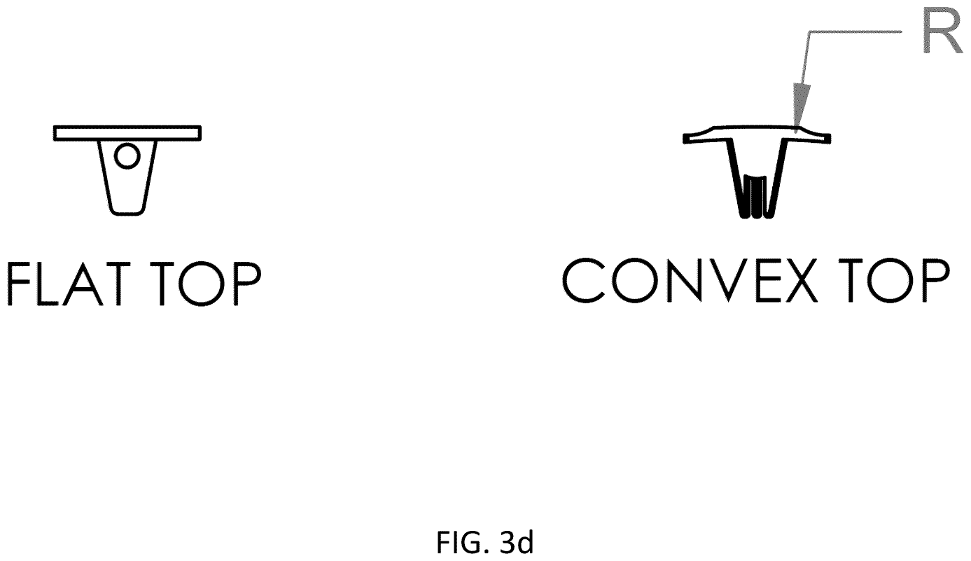

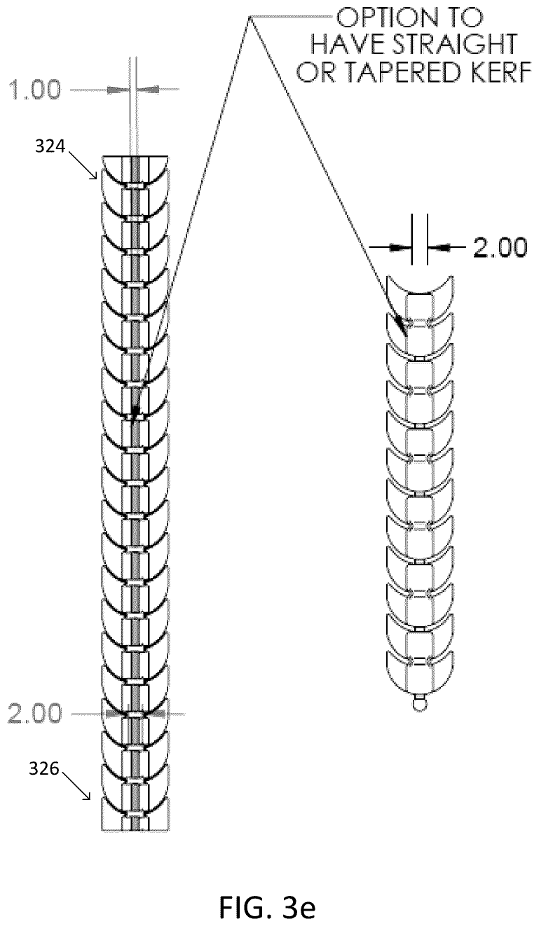

In the example of FIGS. 2a-e, the flexible spine 206 is a single piece spine. In other implementations, the flexible spine may be multi-piece. For example, FIGS. 3a-e show another example of an osteotomy gap filling implant 300 in which the flexible spine is a multi-piece spine 306. More particularly, in this example, the flexible spine is a series of rigid spine segments 316 joined to one another at articular joints 318 (e.g. ball and socket joints). As shown in FIGS. 3a-e, the ball and socket joints 318 allow segments to be added or subtracted from the implant with various accessory segments. For example, segments 320 including bur hole covers with ports may be included and segments 322 with inserts may be included (including fully embedded inserts located within the interior of the segment body and/or partially embedded inserts that extend beyond the segment body). In the example of a craniotomy it is typical to create multiple burr holes using a cranial perforator. Then a routing bit connects the holes. The inserts are intended to allow the device's assembly into the configuration needed to meet the individual needs of the patient. Distance between the burr holes changes and so a longer or shorter segment can be selected to facilitate a complete closure.

As shown in FIG. 3e, the implant may be provided in straight or tapered kerf options. In the example shown on the left in FIG. 3e, the lower bodies at one end 324 of the implant are narrower in width than the lower bodies at the other end 326. More specifically, in the example on the left, the lower bodies taper in width from a first end 326 of the implant to a second end 324 of the implant. In the example on the right, the lower bodies have a uniform width along the length of the implant.

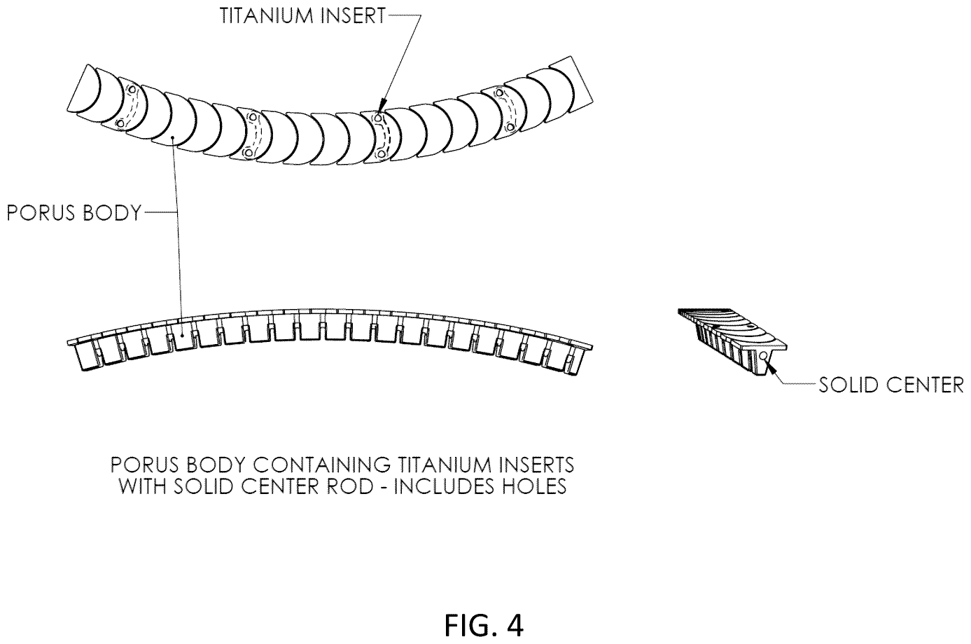

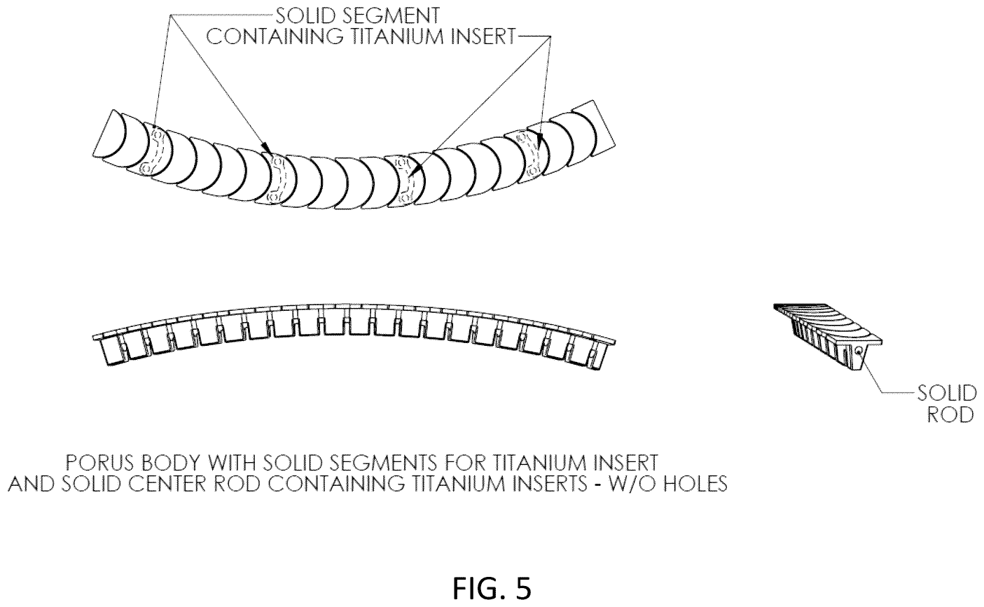

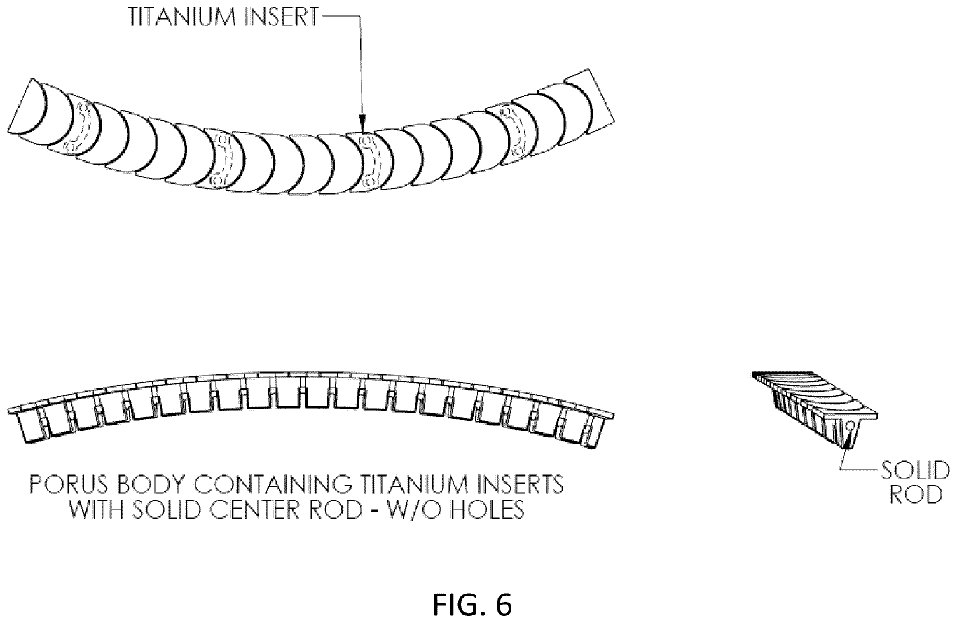

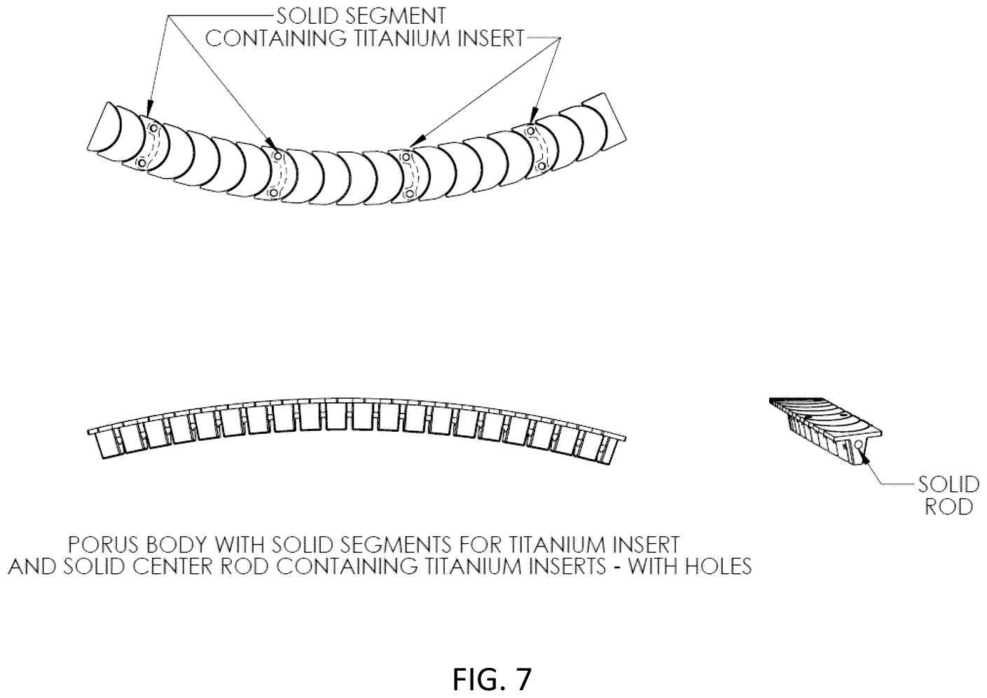

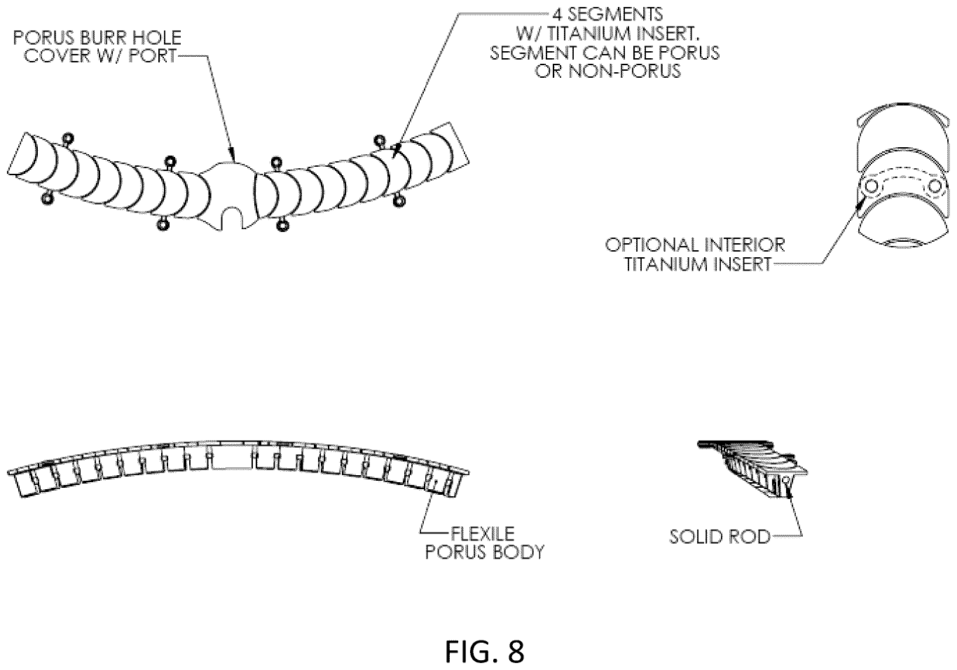

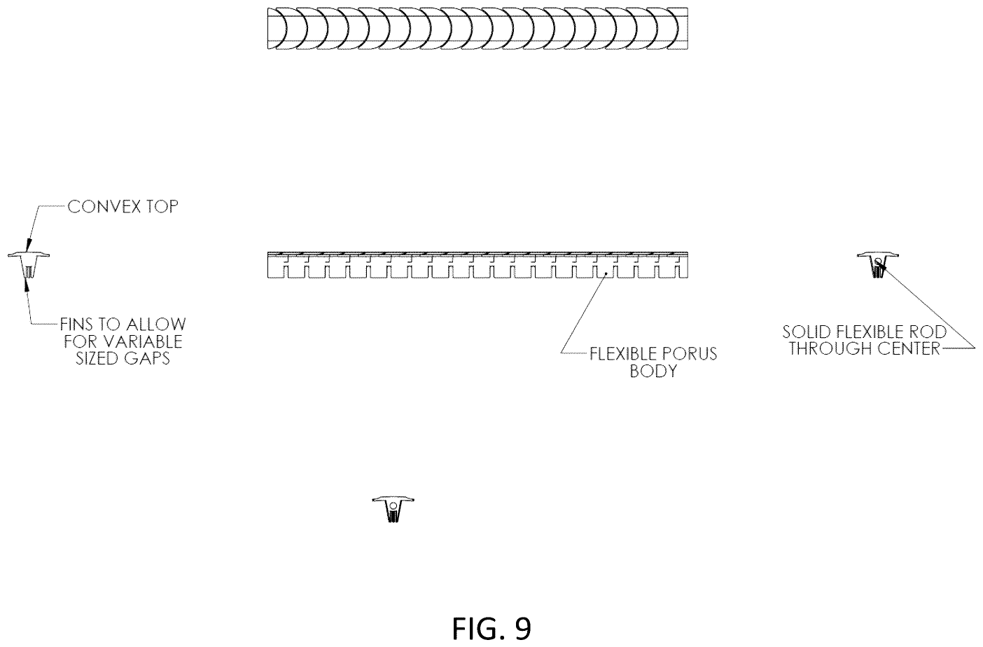

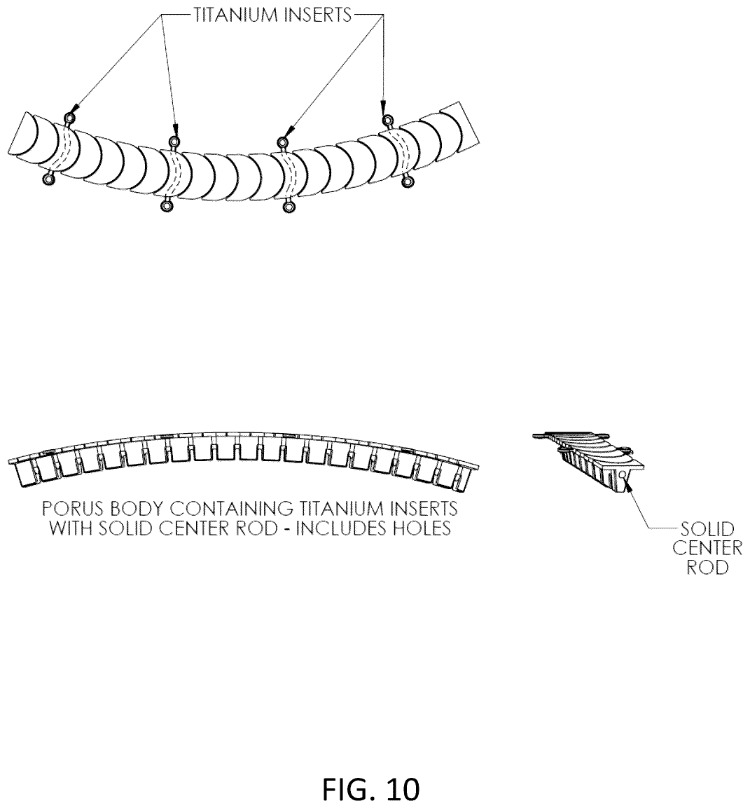

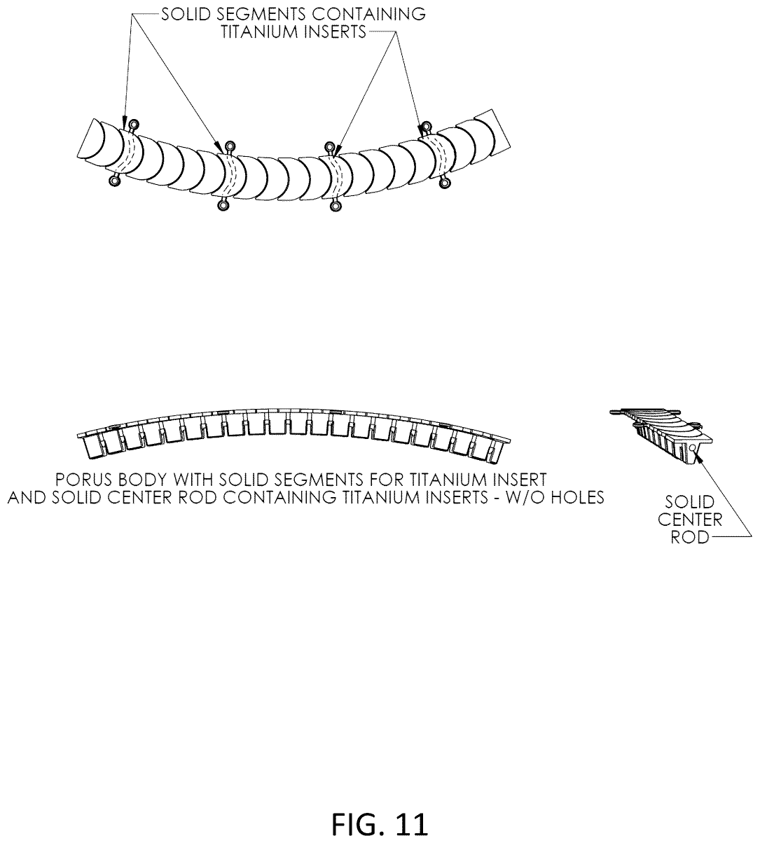

FIGS. 4-12 show additional examples of implants. In the examples of FIGS. 4 and 6, the implant includes porous bodies, some of which include titanium inserts, with a non-porous center rod. The holes in the inserts may be used to receive fixation screws that provide a strong reinforced point of fixation, spacing of these fixation bars that are insert molded into the device are presented typical to the spacing of traditional cranial closure spacing in one example. It is important to note that the articulating top segments surround the titanium. These may be sintered porous material or non-porous material. In the example of FIGS. 5 and 7, the implant includes some segments with porous bodies and other segments (the segments with the inserts) with non-porous bodies. The inserts in this example can be presented where the holes are completely embedded into the material. The titanium plate has holes in its ends allowing for a screw to be inserted yet the holes remain covered with the material unless needed by the surgeon. This leaves the surface smooth and covers the titanium bars. The holes are easy to see in the device and accessed by passing a self-drilling screw into the inner hole of the titanium and through the polymer coating.

In some implementations, the implant's spine may be configured to elongate, in some instances up to 100%, up to 200%, up to 300%, or more, which may enhance the implant's ability to flex and conform to a wide range of shapes and fits.

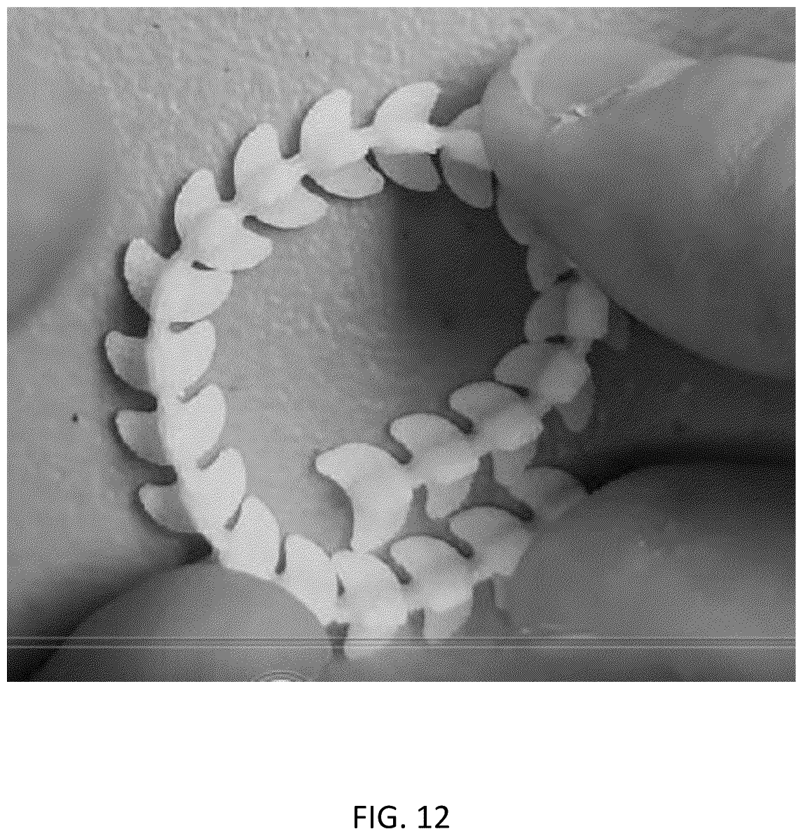

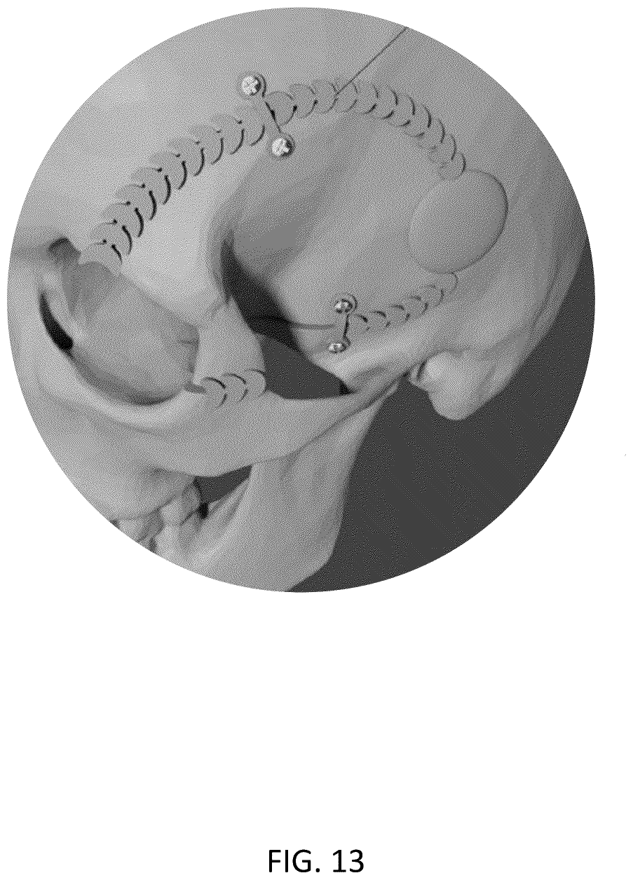

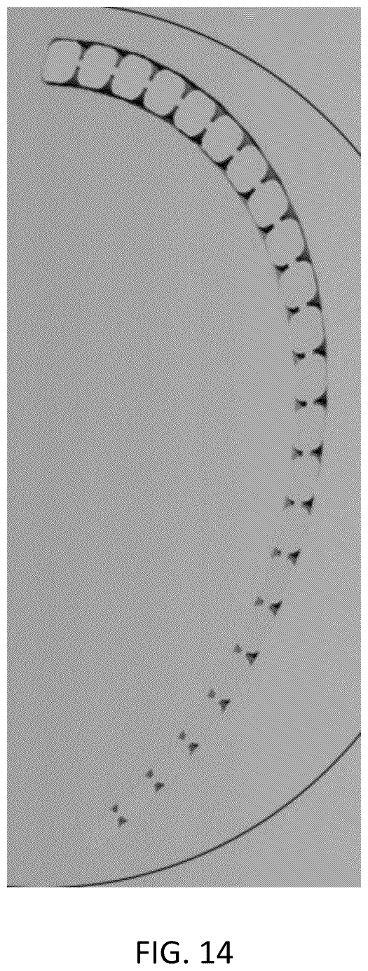

The examples of implants described above are configured to allow for significant amounts of flexion in multiple degrees of freedom without buckling of the implant, allowing the implant to conform to an extremely wide range of shapes to fit a wide range of bone gaps created by osteotomy procedures. FIG. 12 shows a photograph of an example implant rolled into a tight circle, which is due to the shape and configuration of the implant's spine and segments, and would not be possible with earlier gap filling implants. FIG. 13 shows examples of implants implanted into cranial bone. FIG. 14 shows a cross section of one of the implants of FIG. 13, showing how the implant can taper in width to match a taper in the kerf when a portion of the cranial bone has been shifted after an osteotomy.

Examples of the present invention have been described herein, including the best mode known to the inventors for carrying out the invention. The invention is susceptible to various modifications and alternative constructions, and exemplary embodiments have been shown and described in detail. Variations of those embodiments, within the spirit of the present invention, may become apparent to those of ordinary skill in the art upon reading the foregoing description. The inventors expect skilled artisans to employ such variations as appropriate, and the inventors intend for the invention to be practiced otherwise than as specifically described herein. Accordingly, it should be understood that there is no intention to limit the invention to the specific form or forms disclosed, but on the contrary, this invention includes all modifications and equivalents of the subject matter recited in the claim appended hereto as permitted by applicable law. Moreover, any combination of the above-described elements in all possible variations thereof is encompassed by the invention unless otherwise indicated herein or otherwise clearly contradicted by context. The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclose. It will be apparent to one of ordinary skill in the art that many modifications and variations are possible in view of the above teachings.

1. An osteotomy gap filling implant, comprising:
(a) a plurality of implant segments, at least some of the segments comprising a porous body; and
(b) a non-porous flexible spine extending through the plurality of implant segments.

2. The osteotomy gap filling implant of claim 1, wherein adjacent implant segments are configured to flex relative to one another about at least one axis.

3. The osteotomy gap filling implant of claim 1, wherein adjacent implant segments are configured to flex relative to one another about multiple axes.

4. The osteotomy gap filling implant of claim 3, wherein the porous bodies of at least some of the implant segments each comprise a top plate and a lower body descending from the top plate.

5. The osteotomy gap filling implant of claim 4, wherein the top plates of the porous bodies comprise a series of radially nested top plates.

6. The osteotomy gap filling implant of claim 4, wherein the non-porous flexible spine extends through the lower bodies.

7. The osteotomy gap filling implant of claim 4, wherein a first top plate of one of the implant segments and a second top plate of an adjacent implant segment are at least partially separated by a curved gap defined by a first curved edge of the first top plate and a second curved edge of the second top plate.

8. The osteotomy gap filling implant of claim 7, wherein the first curved edge comprises a convex curved edge and the second curved edge comprises a concave curved edge corresponding in shape to the convex curved edge.

9. The osteotomy gap filling implant of claim 4, wherein a first lower body of one of the implant segments and a second lower body of an adjacent implant segment are at least partially separated by a vertical break.

10. The osteotomy gap filling implant of claim 9, wherein the vertical break between the first and second lower bodies defines a flexural gap between the first and second lower bodies.

11. The osteotomy gap filling implant of claim 4, wherein at least one of the lower bodies at a first end of the implant has a narrower width relative to one of the lower bodies at a second end of the implant.

12. The osteotomy gap filling implant of claim 4, wherein the lower bodies taper in width from a first end of the implant to a second end of the implant.

13. The osteotomy gap filling implant of claim 1, wherein the flexible spine comprises a single piece spine.

14. The osteotomy gap filling implant of claim 1, wherein the flexible spine comprises a multi-piece spine.

15. The osteotomy gap filling implant of claim 14, wherein the multi-piece spine comprises a plurality of rigid spine segments joined at articular joints.

16. The osteotomy gap filling implant of claim 15, wherein the articular joints comprise ball and socket joints.

17. The osteotomy gap filling implant of claim 1, wherein at least one of the implant segments further comprises an insert.

18. The osteotomy gap filling implant of claim 17, wherein the segment with the insert has either a porous body or a non-porous body.

19. The osteotomy gap filling implant of claim 17, wherein the insert is either entirely embedded in the segment or partially embedded in the segment.

20. The osteotomy gap filling implant of claim 17, wherein the insert either comprises holes or does not comprise holes.

21. The osteotomy gap filling implant of claim 17, wherein the insert is metal and the implant segment is plastic.

22. The osteotomy gap filling implant of claim 1, wherein at least one of the implant segments comprises a burr hole cover with a top plate that is larger than top plates of adjacent implant segments.

23. The osteotomy gap filling implant of claim 1, wherein at least some of the implant segments are sintered material molded to the non-porous flexible spine.

24. The osteotomy gap filling implant of claim 1, wherein the implant is a cranial osteotomy gap filling implant.

25. The osteotomy gap filling implant of claim 1, wherein the spine is configured to be elongated in length by at least 100% without breaking.

26. The osteotomy gap filling implant of claim 1, wherein the spine is configured to be elongated in length by at least 300% without breaking.

27. An osteotomy gap filling method comprising:
(a) performing a cranial osteotomy in a cranial bone to form a kerf in the cranial bone;
(b) implanting an osteotomy gap filling implant into the kerf, the implant comprising:
(i) a plurality of implant segments, at least some of the segments comprising a porous body; and
(ii) a non-porous flexible spine extending through the plurality of implant segments.

28. The method of claim 27 wherein forming the kerf comprises forming a curved kerf in the cranial bone.

29. The method of claim 28 wherein adjacent implant segments are configured to flex relative to one another about multiple axes such that the implant can be curved to match the curved kerf in the cranial bone.

30. The method of claim 29, wherein the porous bodies of at least some of the implant segments each comprise a top plate and a lower body descending from the top plate, wherein the top plates of the porous bodies comprise a series of radially nested top plates.

31. The method of claim 29 wherein during curving the implant to match the curved kerf, the spine is configured to be elongated in length by at least 100% without breaking.

32. The method of claim 29 wherein during curving the implant to match the curved kerf, the spine is configured to be elongated in length by at least 300% without breaking.

33. The method of claim 28 further comprising, prior to implanting the osteotomy gap filling implant into the kerf, repositioning a portion of the cranial bone such that the kerf tapers from a wider end to a narrower end.

34. The method of claim 33 wherein the porous bodies of at least some of the implant segments each comprise a top plate and a lower body descending from the top plate, wherein at least one of the lower bodies at a first end of the implant has a narrower width relative to one of the lower bodies at a second end of the implant, and wherein the implant is implanted into the kerf such that the first end of the implant is proximate the narrower end of the kerf and the second end of the implant is proximate the wider end of the kerf.
